(19) United States
(12) Patent Application Publication
(10) Pub. No.: US 2019/0108469 A1
(43) Pub. Date: Apr. 11, 2019

(54) SCHEDULE MANAGEMENT SYSTEMS AND METHODS

(71) Applicant: ConnectRN, Inc., Newton, MA (US)

(72) Inventors: Idriz Limaj, Needham, MA (US); Michael Wood, Newton, MA (US)

(21) Appl. No.: 16/154,170

(22) Filed: Oct. 8, 2018

Related U.S. Application Data
(60) Provisional application No. 62/570,159, filed on Oct. 10, 2017.

(51) Int. Cl. G06Q 10/06; G06Q 10/10; G06Q 50/00

(52) U.S. Cl. CPC G06Q 10/063116; G06Q 50/01; G06Q 10/109; G06Q 10/063114

(57) ABSTRACT

Disclosed are various embodiments for facilitating the management of a large network of workers and employers. A schedule management system can includes a registration and verification system that allows a worker to provide his or her resume, certification(s), skills, specialties, experience, contact information, references, and/or any other information that may be needed for creating a worker profile. The schedule management system of the present disclosure can match registered and verified workers with the open shifts of employers.

### CROSS-REFERENCE TO RELATED APPLICATIONS

This application claims priority to, and the benefit of, co-pending U.S. provisional application entitled “SCHEDULE MANAGEMENT SYSTEMS AND METHODS” having Ser. No. 62/570,159, filed on Oct. 10, 2017, which is hereby incorporated by reference in its entirety.

### BACKGROUND

Staff management relates to the scheduling and staffing of workers within a particular workplace. In the nursing industry, the appropriate staffing and scheduling of nurses for a particular healthcare workplace (e.g., hospital, home healthcare, nursing home etc.) is important in the safety and quality of care of the patients cared for in the particular workplace. Typically, a hospital, for example, staffs and schedules shifts as needed from a pool of nurses employed by the hospital.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a schematic block diagram of a networked environment according to various embodiments of the present disclosure.

FIGS. 2A-2E are pictorial diagrams of example user interfaces rendered by a worker client device in the networked environment of FIG. 1 according to various embodiments of the present disclosure.

FIGS. 3A-3B are pictorial diagrams of example user interfaces rendered by the worker client device in the networked environment of FIG. 1 according to various embodiments of the present disclosure.

FIG. 4 is a flowchart illustrating one example of functionality implemented as portions of the schedule management system executed in a computing environment in the networked environment of FIG. 1 according to various embodiments of the present disclosure.

FIG. 5 is a flowchart illustrating one example of functionality implemented as portions of the schedule management system executed in a computing environment in the networked environment of FIG. 1 according to various embodiments of the present disclosure.

FIG. 6 is a flowchart illustrating one example of functionality implemented as portions of the schedule management system executed in a computing environment in the networked environment of FIG. 1 according to various embodiments of the present disclosure.

FIGS. 7A and 7B are flowcharts illustrating examples of functionality implemented as portions of the schedule management system executed in a computing environment in the networked environment of FIG. 1 according to various embodiments of the present disclosure.

FIG. 8 is a flowchart illustrating one example of functionality implemented as portions of the schedule management system executed in a computing environment in the networked environment of FIG. 1 according to various embodiments of the present disclosure.

FIG. 9 is a flowchart illustrating one example of functionality implemented as portions of the schedule management system executed in a computing environment in the networked environment of FIG. 1 according to various embodiments of the present disclosure.

FIG. 10 is a flowchart illustrating one example of functionality implemented as portions of the schedule management system executed in a computing environment in the networked environment of FIG. 1 according to various embodiments of the present disclosure.

FIG. 11 is a schematic block diagram that provides one example illustration of a computing environment employed in the networked environment of FIG. 1 according to various embodiments of the present disclosure.

### DETAILED DESCRIPTION

The present disclosure relates to various systems and methods for facilitating the management of a large network of workers and employers. According to various embodiments of the present disclosure, a worker (e.g., nurse) can easily and efficiently manage his or her schedule with multiple employers at multiple locations via his or her mobile device. According to various embodiments of the present disclosure, an employer (e.g., hospital, homecare, nursing home, etc.) can have on-demand access to a large network of workers that can be matched to the specialties and experience of open shifts at the employer's workplace.

The present disclosure provides a centralized credentialing and verification process for individual workers. Thus, the redundancy workers can experience by submitting credentials to individual workers and the time it takes to submit and wait for verification from each employer/workplace is reduced. Additionally, the employer's costs associated with overtime and placement sites are reduced by being able to select from a verified pool of workers that is greater than the typical pool of workers for a single employer.

In the healthcare industry, staffing involves matching workers of appropriate skills and expertise with the needs of recipients of the worker's services. Scheduling involves estimating an appropriate number of workers to schedule for a particular shift to meet the needs of and provide quality care to the recipients of the workers' services. Typically, a healthcare employer staffs and schedules nurses within a pool of nurses associated with the particular healthcare employer. In some instances, healthcare employers can use a per diem pool of nurses to select from such that the nurses can be selected to work on an “as needed” basis. In situations where the demand for certain skilled and/or experienced nurses is high for a particular healthcare workplace (e.g., disease outbreak, accidents resulting in multiple injured patients, holidays, etc.), the pool of employed nurses can be limiting. This can directly affect the ability to deliver the quality care needed for patients of the healthcare workplace. Additionally, in situations where the demand for nurses may be low, nurses can be left without a shift opportunity.

The systems and methods of the present disclosure relate to a schedule management system that can expand the pool of workers that an employer may select from to satisfy staffing and scheduling needs while reducing (1) the overhead of worker management at each employer/workplace and (2) the credential and skills verification of the workers. Additionally, the schedule management system of the present disclosure can increase flexibility and control by the workers by expanding the pool of employers and shift opportunities for a worker.

According to various embodiments, the schedule management system can include a registration and verification system that is accessible via a worker's mobile device and allows a worker to seamlessly provide his or her resume, certification(s), skills, specialties, experience, contact information, references, and/or any other information that may be needed for creating a worker profile. For example, a worker can register and provide the appropriate information to create his or her profile through a series of user interfaces rendered via his or her mobile device. In some embodiments, the series of user interfaces are rendered using a mobile onboarding structure. In some embodiments, the registration and verification system may provide incentives (e.g., reward points, monetary incentives, etc.) to encourage a worker to continue through the series of user interfaces to complete the registration process.

In some embodiments, the worker can take a picture of a particular document (e.g., resume, certification, driver's license, etc.) via the camera on his or her mobile device and upload the image via a user interface rendered on the mobile device. The registration and verification system can convert the image to a readable text and then parse and extract the appropriate information from the uploaded image(s). In various embodiments of the present disclosure, the worker's profile can be automatically verified, thereby reducing the overhead and time that an employer would spend prior to employing a particular worker. In some embodiments, manual review may occur in addition to the automated process. In some embodiments, the worker's profile can be updated according to employer feedback, experience, and/or any other factor as can be appreciated.

According to various embodiments, the schedule management system of the present disclosure can match registered and verified workers with the open shifts of one or more employer(s). The workers can be matched with a shift according to a variety of factors, such as, for example, skill, specialty, experience, level, distance between worker location and workplace location, worker preference, employer preference, type of workplace, worker similarities, facility similarities (e.g., the worker has received positive and/or negative feedback from similar types of facilities), and/or any other factor as can be appreciated.

In some embodiments, the schedule management system can identify skills and/or experiences of the worker that may not be provided by the worker. For example, a worker may have worked in a cardiac unit, but may not have provided information indicating that he or she has cardiac experience. The schedule management system can infer the cardiac experience based on the type of facility that the worker has previously worked.

According to various embodiments, a worker can view and select open shifts via one or more user interfaces rendered on his or her mobile device. In some embodiments, the schedule management system can transmit notification alerts to the worker with respect to scheduled shifts. The notification alerts can be in the form of an audio alert, a text message, a push notification, a visual alert, and/or any other type of alert as can be appreciated. According to various embodiments, an employer can post open shifts, accept and/or decline particular workers for certain shifts, provide feedback for workers, and/or any other function as can be appreciated.

According to various embodiments, the schedule management system can employ machine learning models to determine appropriate scheduling and staffing needs according to historical data including, but not limited to, prior staffing needs, prior scheduling needs, weather conditions, seasons (e.g., fall, winter, spring, summer, holidays, etc.), shift cancellations, number of open shifts, historical worker data, and/or any other factor as can be appreciated.

As one skilled in the art will appreciate in light of this disclosure, certain embodiments may be capable of achieving certain advantages that improve the functioning of computer systems and the functioning of employer and worker network scheduling, including some or all of the following: (1) improved flexibility to generate a verified network of workers using a mobile onboarding experience; (2) dynamic ability to adjust in real time based on varied client conditions; (3) dynamic ability to adjust in real time based on varied employer facility conditions; (4) improved computing efficiencies by, for example, reducing processing demands by centralizing the verification, registration, and scheduling system; (5) improved employer facility operations; (6) reduction of worker and employer costs; and so on.

In the following discussion, a general description of the system and its components is provided, followed by a discussion of the operation of the same.

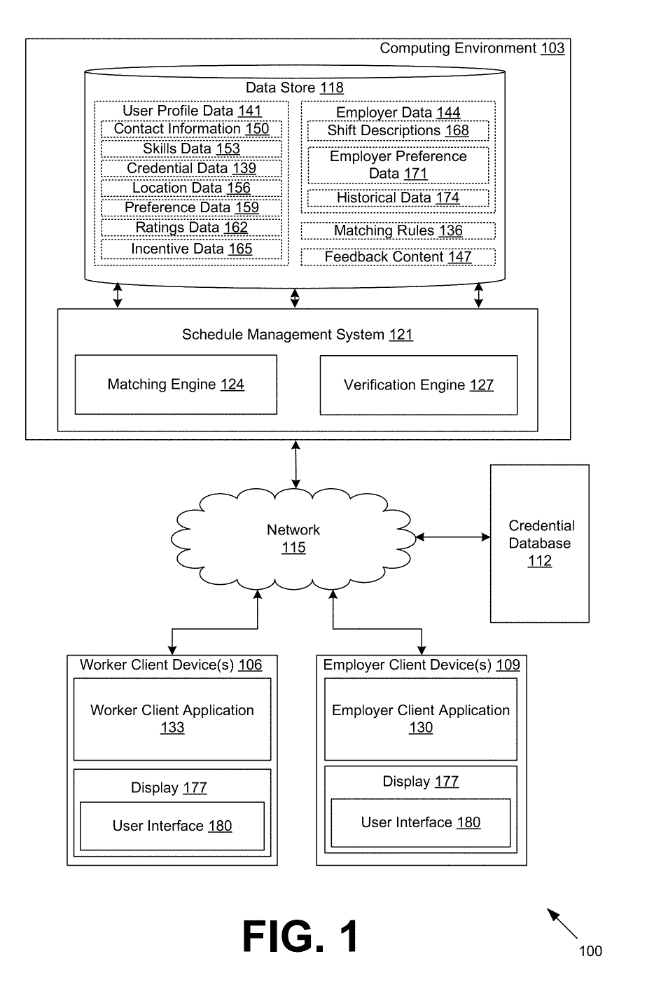

With reference to FIG. 1, shown is a networked environment 100 according to various embodiments. The networked environment 100 includes a computing environment 103, a worker client device 106, an employer client device 109 and a credential database 112, which are in data communication with each other via a network 115. The network 115 includes, for example, the Internet, intranets, extranets, wide area networks (WANs), local area networks (LANs), wired networks, wireless networks, cable networks, satellite networks, or other suitable networks, etc., or any combination of two or more such networks.

The computing environment 103 may comprise, for example, a server computer or any other system providing computing capability. Alternatively, the computing environment 103 may employ a plurality of computing devices that may be arranged, for example, in one or more server banks or computer banks or other arrangements. Such computing devices may be located in a single installation or may be distributed among many different geographical locations. For example, the computing environment 103 may include a plurality of computing devices that together may comprise a hosted computing resource, a grid computing resource, and/or any other distributed computing arrangement. In some cases, the computing environment 103 may correspond to an elastic computing resource where the allotted capacity of processing, network, storage, or other computing-related resources may vary over time.

Various applications and/or other functionality may be executed in the computing environment 103 according to various embodiments. Also, various data is stored in a data store 118 that is accessible to the computing environment 103. The data store 118 may be representative of a plurality of data stores 118 as can be appreciated. The data stored in the data store 118, for example, is associated with the operation of the various applications and/or functional entities described below.

The components executed on the computing environment 103, for example, include a schedule management system 121, a matching engine 124, a verification engine 127, and other applications, services, processes, systems, engines, or functionality not discussed in detail herein. The schedule management system 121 is executed to facilitate the management and scheduling of shifts between a network of workers and a network of employers. For example, the schedule management system 121 is executed to be an interface between the employer client application 130 executed on the employer client device 109 and the worker client application 133 executed on the worker client device 106.

The matching engine 124 is executed to match workers with open shifts according to a variety of attributes including, for example, skills, experience, prior ratings, worker preferences, employer preferences, location of shift facility, worker similarities, and/or any other attributes as can be appreciated. The matching engine 124 may employ matching rules 136 which may be used to define weights that may be assigned to the various factors. The weights may be used to generate a score for a particular worker. In some embodiments, some attributes may be assigned a greater weight than other attributes. For example, skills may be given a greater weight than location of shift facility.

The verification engine 127 may be executed to verify information received from the worker client device 106. For example, the verification engine 127 may be configured to interface with the credential database 112 to verify that the credential data 139 submitted by a particular worker is up-to-date and valid. The credential database 112 may comprise a third-party database that includes public information about individuals. For example, various states may have state-supported databases that include public information including credential information that the verification engine 127 can access in verify the information provided by the worker is valid.

The data stored in the data store 118 includes, for example, user profile data 141, employer data 144, matching rules 136, feedback content 147, and potentially other data. The user profile data 141 includes profile data for each of the workers included in the network of workers associated with the schedule management system 121 of the present disclosure. The user profile data 141 may include contact information 150, skills data 153, credential data 139, location data 156, preference data 159, ratings data 162, incentive data 165, and/or any other data as can be appreciated.

The contact information 150 may include, for example, mailing address, residential address, email address, telephone contact information, and/or any other information as can be appreciated. The skills data 153 may include self-reported skills and/or specialties of the particular worker. For example, the skills data 153 may include experience data, level title, specialties, and/or other skill information as can be appreciated. For example, the skills data 153 may include a level title associated with the worker. The different levels for a nurse include a registered nurse (RN), certified nursing assistant (CNA), a licensed practical nurse (LPN), a nurse technician, and/or any other level as can be appreciate. The skills data 153 can further include specialties associated with the worker. For example, specialties for a nurse may include, critical care, dialysis, emergency room, geriatric, home health, hospice, neurology, orthopedics, operating room, oncology, pediatrics, psychiatry, labor & delivery, gynecologic, etc.

The skills data 153 can further include experience data obtained from a resume and/or other document provided by the worker. In some embodiments, the experience data may be extracted from an image of a resume and/or other document provided by the worker client device 106. For example, the worker may capture a picture of his or her resume and/or other document via a camera of the worker client device 106 and send the picture of the resume to the schedule management system 121. In other embodiments, the worker may send a copy of a resume and/or other document in another form (e.g., PDF, .doc, .txt, etc.) and the experience data may be populated by extracting the information from the document image uploaded to the computer environment 103.

The credential data 139 can include information related to the credentials of the worker. For example, the credential information may include one or more certificates and/or registrations associated with the particular worker. For example, for a nurse, the credential data may include a RN/LPN/CNA registration image, RN/LPN/CNA registration data, CPR certifications, ACLS Certification, and/or any other type of certification and/or registration as can be appreciated. The credential data 139 may further comprise expiration dates associated with the particular registrations and/or certificates.

The location data 156 can include data associated with the location of the worker client device 106. For example, the worker client device 106 can send location data 156 to the schedule management system 121 notifying the system of a current location of the worker client device 106. In some embodiments, the location data 156 is provided in response to a request from the schedule management system 121. In other embodiments, the worker client device 106 may periodically transmit location data 156 to the schedule management system 121. The location data 156 may correspond to GPS (global positioning system) coordinates obtained via a GPS device of the worker client device. In some embodiments, the location data 156 can be inputted by a user via a user interface associated with the worker client application 133. The location data 156 can be used by the matching engine 124 as a factor in matching a particular worker with a particular open shift.

The preference data 159 can include preferences provided by the worker with respect to particular open shift opportunities. For example, the preference data 159 can include a distance (e.g., a radius) a worker is willing to travel for a particular shift. The preference data 159 can further include a preference associated with a particular time range associated the shift. In addition, a worker can provide a ranking of preferred types of employment, skills, and/or specialty areas, that the worker would prefer to work. For example, a worker may prefer to work in an emergency room over a critical care unit.

The ratings data 162 can include ratings for the particular worker as provided by prior employers. For example, following a worker's completion of a shift, the employer may provide feedback. The ratings data 162 can include an overall rating, ratings for different skills, and/or any other type of rating as can be appreciated. The incentive data 165 can include points accumulated by a particular user during use of the application. For example, as a user completes various levels of the mobile onboarding registration process, the user may be awarded points to incentivize the user to continue with the onboarding registration process. In some embodiments, a worker can access a third-party system to redeem the points for a monetary value (e.g., gift card, equipment, etc.). For example, a user interface rendered on the worker client device 106 may contain a link to a third-party website which allows the user to redeem his or her accumulated points.

The employer data 144 includes data associated with the employer. For example, the employer data 144 can include shift descriptions 168, employer preference data 171, historical data 174, and/or any other data as can be appreciated. The shift descriptions 168 can correspond to available open shifts. The shift descriptions 168 can include the skills required, experience required, level of worker required, time and date of shift, location of shift, and/or any other data as can be appreciated.

In some embodiments, the employer preference data 171 can include employer preferences for particular job descriptions and/or shifts. In one non-limiting example, the employer may prefer that certain open shifts be presented to a certain pool of workers in the network before being presented to a larger pool of workers. For example, the employer may want to present the open shifts to their own employees prior to opening up the shifts to other nurses not directly employed.

The historical data 174 can include data associated each of the employers over a period of time. The historical data 174 can be employed by machine learning models and used to determine whether an employer should overstaff particular shifts according to the historical data. For example, the historical data 174 can be used to predict a number of cancellations by workers for a particular day according to learned data including past weather patterns and predicted weather patterns for the particular day.

The matching rules 136 can comprise rules associated with matching open shifts with workers. For example, the matching rules 136 may comprise algorithms that can be applied to match particular workers with available shifts. In some embodiments, the matching rules 136 can include rules that indicate weights to assign to various factors (e.g., location, skills, experience, rating, preference, etc.) associated with the worker and/or the open shift requirements. For example, a score can be calculated and be assigned to a particular worker according to a sum of weights. If a score associated with the user for a particular open shift is within a predefined threshold, the particular open shift can be presented to the user. However, if the score associated with the user is outside of a predefined threshold, the particular open shift may not be presented to the user.

In other embodiments, the matching rules 136 can comprise ranking rules. For example, assume multiple workers are considered for a particular open shift. Each worker may be assigned a score based on a sum of weights assigned to one or more factors. The workers can be ranked according to his or her respective score. The matching rules 136 can indicate a predefined percentage, predefined ratio and/or predefined number of top-ranked workers to select for consideration for the particular open shift.

The feedback content 147 can include content and/or rules relating to feedback received from the employer with respect to a particular worker and/or received from the worker with respect to a particular employer. For example, the feedback content 147 can include questions that can be presented to an employer. Each of the questions may have weights associated with them with respect of a level of significance in an overall rating determination. For example, a question asking what tasks the worker completed during a shift may not be weighted as high as a question regarding the worker's overall performance. The feedback information obtained can be used for future placement of the worker and/or other workers that are determined to be similar to the worker according to his or her respective attributes, credentials, ratings, etc.

The worker client device(s) 106 and the employer client device(s) 109 are each representative of a plurality of client devices that may be coupled to the network 115. The worker client device 106 and the employer client device 109 may each comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, smartwatches, head mounted displays, voice interface devices, or other devices. The worker client device 106 and the employer client device 109 may each include a display 177. The display 177 can comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc.

The worker client device 106 may be configured to execute various applications, such as a worker client application and/or other applications. The worker client application 133 can be executed in a worker client device 106, for example, to access network content served up by the computing environment 103 and/or other servers, thereby rendering a user interface on the display. To this end, the worker client application 133 can comprise, for example, a browser, a dedicated application, etc., and the user interface 180 may comprise a network page, an application screen, etc. The worker client device 106 may be configured to execute applications beyond the worker client application 133 such as, for example, email applications, social networking applications, word processors, spreadsheets, and/or other applications.

The employer client device 109 may be configured to execute various applications, such as an employer client application 130 and/or other applications. The employer client application 130 can be executed in an employer client device 109, for example, to access network content served up by the computing environment 103 and/or other servers, thereby rendering a user interface 180 on the display. To this end, the employer client application 130 may comprise, for example, a browser, a dedicated application, etc., and the user interface may comprise a network page, an application screen, etc. The employer client device 109 may be configured to execute applications beyond the worker client application such as, for example, email applications, social networking applications, word processors, spreadsheets, and/or other applications.

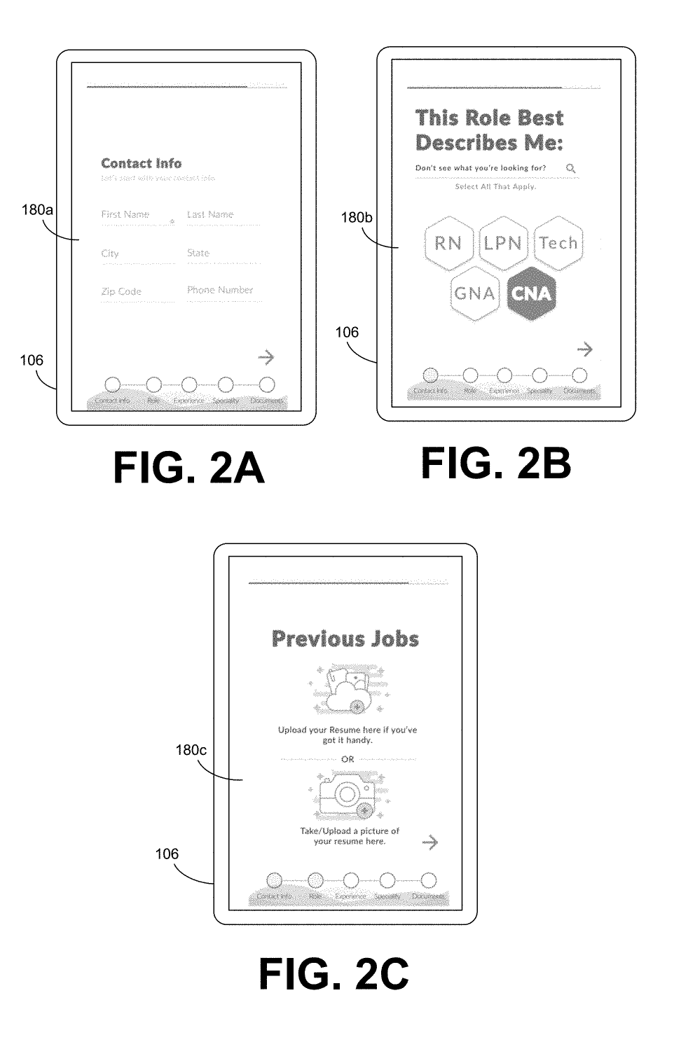

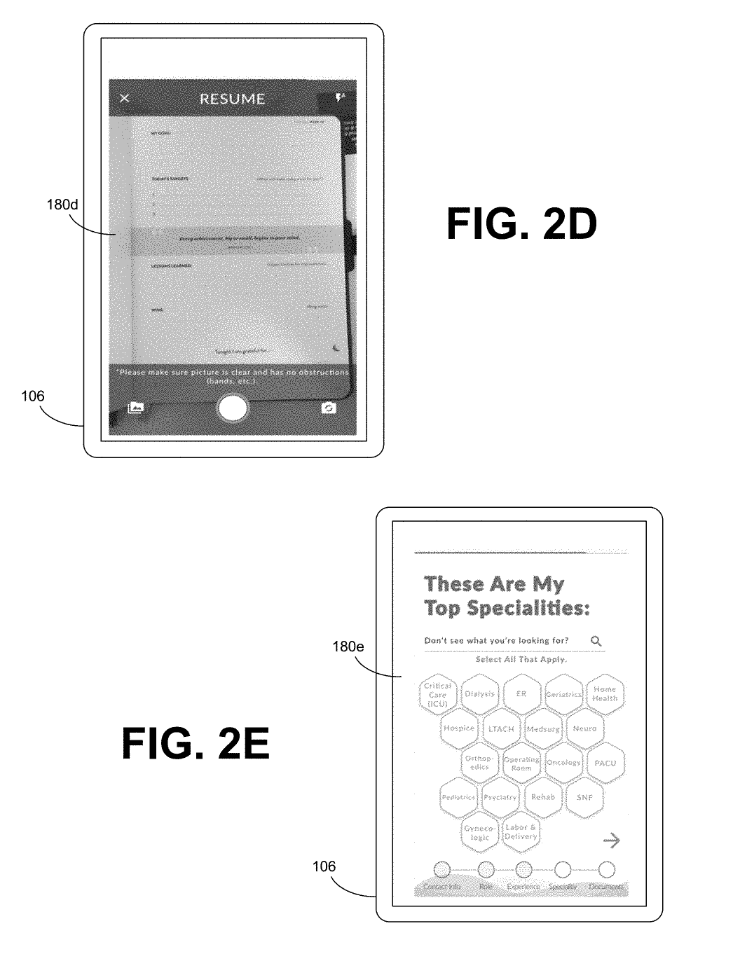

Referring next to FIGS. 2A-2E and 3A-3B, shown are example user interfaces 180 (e.g., 180a-180g) that may be rendered on the worker client device 106 according to various embodiments of the present disclosure. In particular, FIGS. 2A-2E and 3A-3B illustrate various user interfaces that a worker may use to register with the program, provide profile information, review and schedule open shifts, and/or manage overall schedule with respect to multiple employers at multiple locations. For example, FIGS. 2A-2E illustrate sample user interfaces 180 (e.g., 180a-180e) that may be included in the series of user interfaces rendered by the worker client device 106 during the onboarding-based registration process. In particular, FIG. 2A illustrates a user interface 180a that allows a user to input contact information. FIG. 2B illustrates an example of a user interface 180b that includes selectable components that allows a user to select an appropriate certification level. Although, FIG. 2B illustrates selectable components, in some embodiments, the user interface can include text entry boxes, buttons, drop-down boxes, and/or any other type of component as can be appreciated.

Turning now to FIG. 2C, shown is an example user interface 180c that facilitates a user to upload a document (e.g., resume) for a stored location and/or take a picture of the document. FIG. 2D illustrates an example user interface 180d that corresponds to the interface with a camera (not shown) of the worker client device 106 for capturing a picture of a document. FIG. 2E illustrates an example user interface 180e that includes components for selecting skills of the worker.

FIGS. 3A-3B illustrate example user interfaces 180 (e.g., 180f and 180g) that can be rendered by the worker client devices 106 with respect to scheduling of shifts and entry of user preferences for the worker. In particular, FIG. 3A illustrates an example user interface 180f rendered on a worker client device 106. The user interface 180f includes selectable components for setting user preferences that can be used by the schedule management system 121 in matching the user with available open shifts. For example, the worker can select time frames in which they are willing to work.

Further, the worker can activate and/or deactivate his or her availability. For example, a worker will not be considered by the schedule management system 121 when his or her availability is deactivated. It should be noted that while FIG. 3A illustrates user provided available time frames, in some embodiments, the schedule management system 121 can extract calendar information from a calendar associated with the user to determine when a user is available. In some embodiments, the worker can be provided a user interface that allows the user to adjust availability from data extracted from a calendar.

FIG. 3B illustrates an example user interface 180g including a notification of an open shift match for a particular user according to various embodiments of the present disclosure. In particular, the user interface 180g of FIG. 3B includes information (e.g., time, facility, supervisor contact information, location, etc.) associated with an open shift as well as selectable components for applying for and/or accepting the particular open shift.

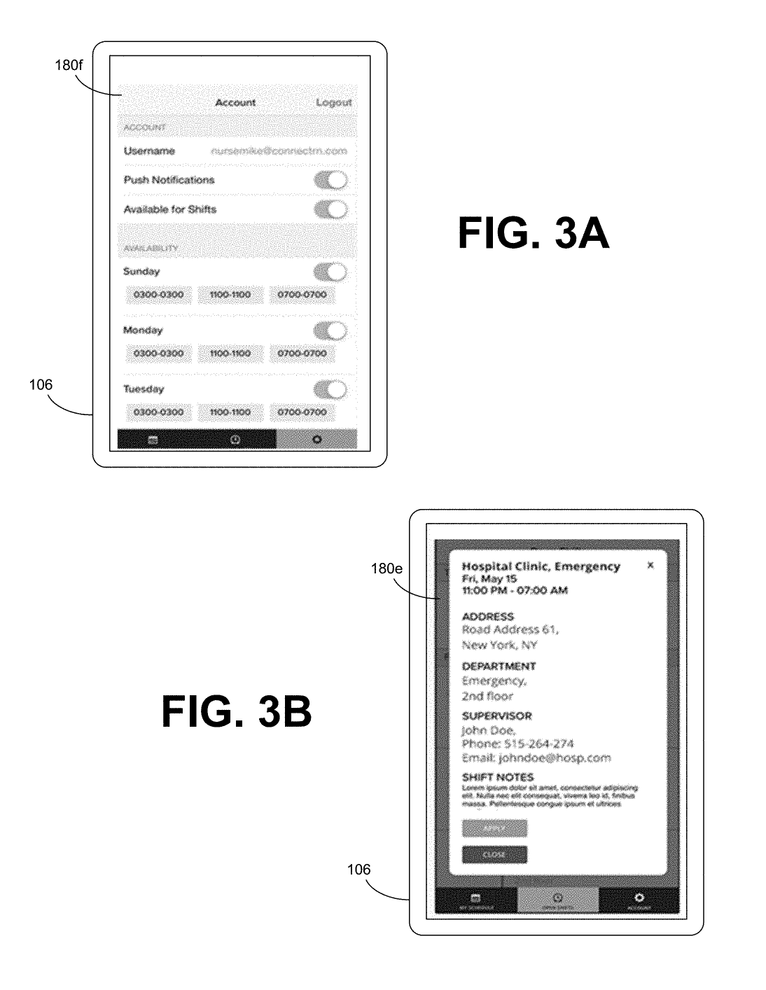

Referring next to FIG. 4, shown is a flowchart that provides one example of the operation of a portion of the schedule management system 121 according to various embodiments. It is understood that the flowchart of FIG. 4 provides merely an example of the many different types of functional arrangements that may be employed to implement the operation of the portion of the schedule management system 121 as described herein. As an alternative, the flowchart of FIG. 4 may be viewed as depicting an example of elements of a method implemented in the computing environment 103 (FIG. 1) according to one or more embodiments.

Beginning with box 400, the schedule management system 121 obtains user contact information 150 (FIG. 1) from a worker client device 106 (FIG. 1). For example, the schedule management system 121 may obtain the user contact information 150 over the network 115 via one or more user interfaces 180 rendered on the worker client device 106. In box 403, the schedule management system 121 obtains skills data 153 (FIG. 1) from the worker via one or more user interfaces 180 rendered on the worker client device 106. The skills data 153 includes self-reported skills and/or specialties of the particular worker. For example, the skills data 153 may include experience data, skill level, specialties, and/or other skill information as can be appreciated.

The skills data 153 may also include a title associated with the worker. The different levels for a nurse include a registered nurse (RN), certified nursing assistant (CNA), a licensed practical nurse (LPN), a nurse technician, and/or any other level as can be appreciate. The skills data 153 can further include specialties associated with the worker. For example, specialties for a nurse may include, critical care, dialysis, emergency room, geriatric, home health, hospice, neurology, orthopedics, operating room, oncology, pediatrics, psychiatry, labor & delivery, gynecologic, etc. The skills data may further include experience data (e.g., resume).

In box 406, the schedule management system 121 obtains credential data 139 (FIG. 1) corresponding to the user. For example, the schedule management system 121 may obtain credential data 139 from the user via one or more user interfaces 180 rendered on the worker client device 106. For example, the credential data 139 may include one or more certificates and/or registrations associated with the particular worker. For example, for a nurse, the credential data 139 may include a RN/LPN/CNA registration image, RN/LPN/CNA registration data, CPR certifications, ACLS Certification, and/or any other type of certification and/or registration as can be appreciated. The credential data 139 can further include one or more expiration dates associated with the particular registrations and/or certificates.

In some embodiments, the schedule management system 121 can be in data communication with a third-party and extract information (e.g., contact data, skills data, credential data from the third party, etc.) associated with the worker. For example, a user can permit the schedule management system 121 to contact with a third-party social media system (e.g., FACEBOOK®, LINKEDIN®, TWITTER®, etc.) and request/receive information from the third-party system that can be used in creating the worker's profile.

In box 409, the schedule management system 121 determines whether any of the data received contains an image (e.g., .jpeg). For example, a user may upload via a user interface 180 an image of his or her resume and/or other type of document containing experience information. If the data does not contain an image, the schedule management system 121 proceeds to box 415. Otherwise, the schedule management system 121 proceeds to box 412. In box 412, the schedule management system 121 converts the image to a text format (e.g., .doc, .pdf, .txt, etc.) and extracts the data associated with the contact information, experience, and/or credentials. For example, if a user uploads an image of his or her resume, the schedule management system 121 can apply algorithms and keywords to parse and identify the data fields associated with the worker's resume. As such, the schedule management system 121 can extract data corresponding to the worker's experience.

In box 415, the schedule management system 121 stores the contact information 150, skills data 153, and credential data 139, including any data extracted from any submitted images, in the data store 118. In box 418, the schedule management system 121 verifies the data. For example, the schedule management system 121 may employ the verification engine 127 to interface with one or more credential databases 112 to verify that the information self-reported by the worker is valid. In some embodiments, the verification engine 127 may periodically request and receive credential information from the one or more credential databases 112 and compare the received information with the user-submitted data.

In some embodiments, the data may be verified by manual review. In box 421, the schedule management system 121 establishes a user account for the user. When the user account is established for the user, the user can begin scheduling shifts with multiple employers at multiple locations via the schedule management system 121. Upon establishing the user account, this portion of the schedule management system 121 ends.

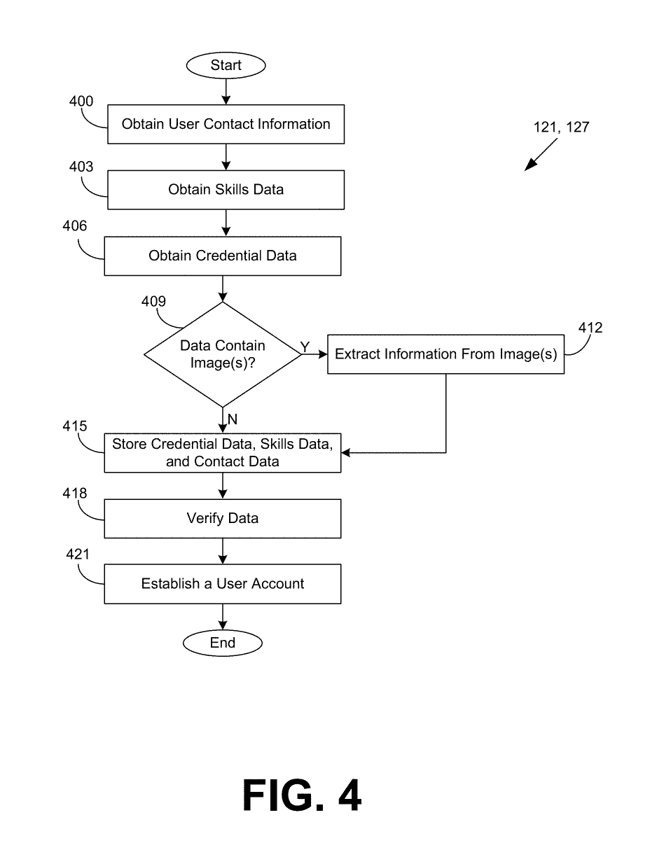

Referring next to FIG. 5, shown is a flowchart that provides one example of the operation of a portion of the schedule management system 121 according to various embodiments. It is understood that the flowchart of FIG. 5 provides merely an example of the many different types of functional arrangements that may be employed to implement the operation of the portion of the schedule management system 121 as described herein. As an alternative, the flowchart of FIG. 5 may be viewed as depicting an example of elements of a method implemented in the computing environment 103 (FIG. 1) according to one or more embodiments.

In box 500, the schedule management system 121 receives an image from the worker client device. For example, a user may take a picture of his or her resume and/or upload the resume image during the registration process. In box 503, the schedule management system 121 may convert the image so that text associated with particular data fields can be extracted from the image according to various embodiments. For example, if the image is in .jpeg format, the image will need to be converted to a text format such as, for example, .txt, .doc, .pdf, and/or any other format as can be appreciated. In box 506, the schedule management system 121 identifies data fields within the image. In one non-limiting example, the schedule management system 121 may parse the document and compare words from the image with a set of predefined keywords. For example, for a resume, the predefined keywords may include “objective,” “experience,” “education,” “activities,” and/or other keywords as can be appreciated. As such, the information that follows these identified keywords can be used to populate the data fields of the worker's profile. In some embodiments, the schedule management system 121 can compare extracted words to synonyms of predefined keywords. In box 509, the schedule management system 121 extracts the data from the image and in box 512, the schedule management system 121 stores the extracted data in the data store 118.

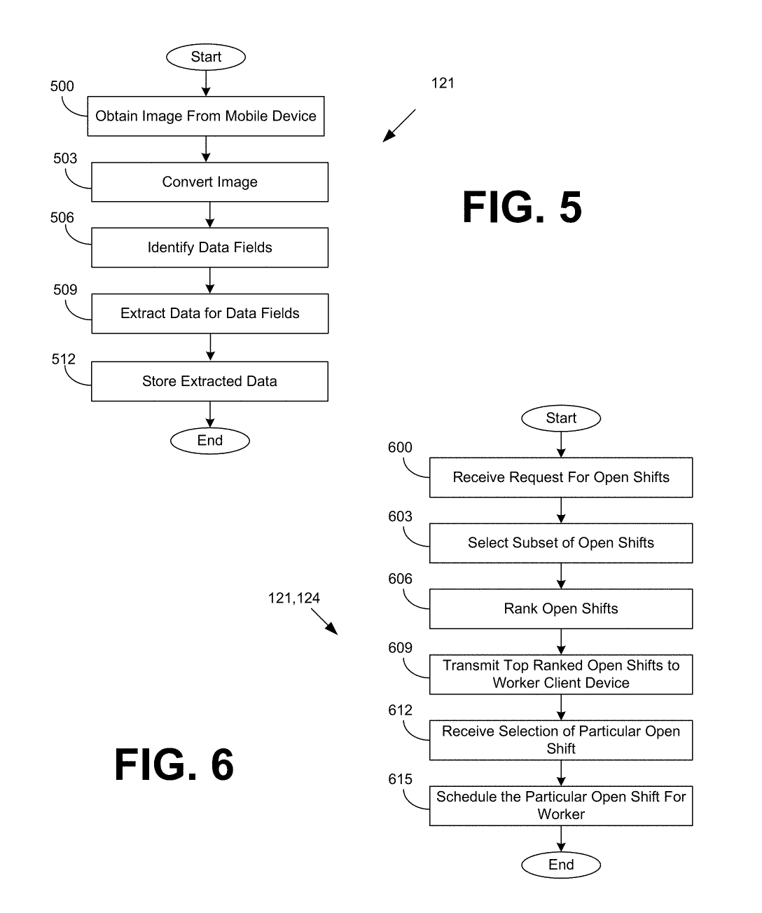

Referring next to FIG. 6, shown is a flowchart that provides one example of the operation of a portion of the schedule management system 121 according to various embodiments. It is understood that the flowchart of FIG. 6 provides merely an example of the many different types of functional arrangements that may be employed to implement the operation of the portion of the schedule management system 121 as described herein. As an alternative, the flowchart of FIG. 6 may be viewed as depicting an example of elements of a method implemented in the computing environment 103 (FIG. 1) according to one or more embodiments.

In box 600, the schedule management system 121 receives a request for open shifts according to various embodiments of the present disclosure. The request may be received from the worker client device 106 via a user interface 180 associated with the worker client application 133. In box 603, matching engine 124, selects a subset of open shifts. The subsets may be selected according to a variety of factors such as, for example, location, skills, specialties, worker preference, employer preference, etc. For example, if the worker is an emergency room nurse, open shifts for emergency room nurses may be selected while open shifts for homecare may not be selected.

In box 606, the matching engine 124 ranks the open shifts. The open shifts may be ranked according to a variety of factors such as, for example, location, skills, specialties, worker preference, employer preference, worker similarities, etc., according to various embodiments of the present disclosure. For example, the matching engine 124 may assign weights to each of the factors and generate a score based at least in part on a sum of the weights. The weights may be defined in the matching rules 136 and can vary according to the type of factor, employer preference, etc. Each open shift may be ranked according to a respective score.

In box 609, one or more top-ranked open shifts are transmitted to the worker client device 106. The one or more top-ranked open shifts may be presented to a user via a user interface 180 rendered on the worker client device 106. In some embodiments, the schedule management system 121 may generate the user interface 180 and encode the user interface 180 with the top-ranked open shifts. In other embodiments, the worker client application 133 may generate the user interface 180 and encode the user interface 180 with the top-ranked open shifts.

In box 612, the schedule management system 121 receives a selection of a particular open shift. For example, a worker may select via a user interface 180 rendered on the worker client device 106 a particular open shift from the display of open shifts matched with the worker. In box 615, the schedule management system 121 schedules the particular open shift for the worker.

In some embodiments, the schedule management system 121 may send a notification to the employer client device 109 notifying the employer that the shift has been scheduled. In some embodiments, the schedule management system 121 may wait to schedule the worker with the open shift until receiving approval from the employer client device 109. Upon scheduling the worker with the open shift, this portion of the schedule management system 121 ends.

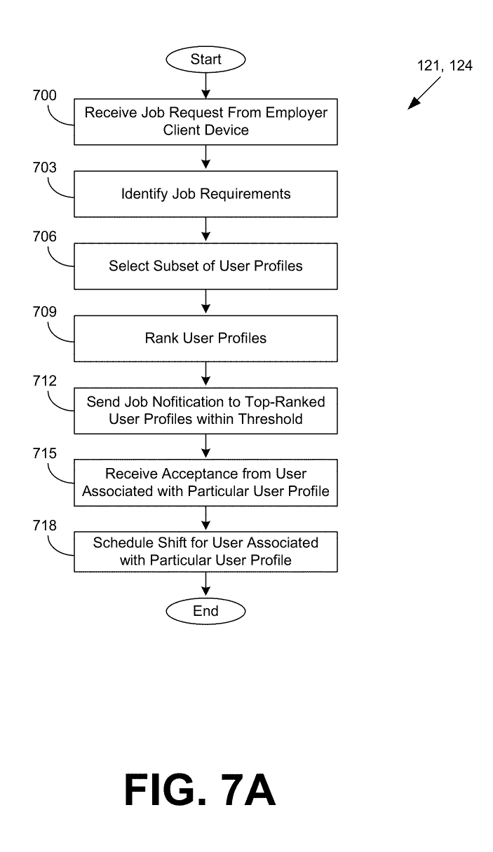

Referring next to FIG. 7A, shown is a flowchart that provides one example of the operation of a portion of the schedule management system 121 according to various embodiments. It is understood that the flowchart of FIG. 7A provides merely an example of the many different types of functional arrangements that may be employed to implement the operation of the portion of the schedule management system 121 as described herein. As an alternative, the flowchart of FIG. 7A may be viewed as depicting an example of elements of a method implemented in the computing environment 103 (FIG. 1) according to one or more embodiments.

In box 700, the schedule management system 121 receives an open shift opportunity from an employer client device 109 according to various embodiments of the present disclosure. The open shift opportunity can be received via a user interface 180 rendered on the employer client device 109. In box 703, the schedule management system 121 determines the requirements of the open shift. For example, the open shift opportunity may specify the skills, specialties, certifications, registrations, and/or other type of information that the employer is looking for in a worker to fill the open shift.

In box 706, the schedule management system 121 via the matching engine 124 selects a subset of user profiles based at least in part on the requirements of the open shift. For example, if the open shift is for a geriatric nurse, a user profile that includes geriatrics as a specialty may be selected while a user profile that includes pediatrics as a specialty may not be selected.

In box 709, the matching engine 124 ranks the user profiles according to a variety of factors such as for example, skills, experience, rating, location, whether the worker is an employee of the employer, worker similarities and/or any other factor as can be appreciated. In some embodiments, the factors are assigned weights and the sum of the weights may be used to rank the user profiles.

In box 712, one or more notifications detailing the open shift are generated and transmitted to the worker client devices 106 of the one or more top-ranked user profiles. In box 715, the schedule management system 121 receives an acceptance of the open shift from a particular worker client device. In box 718, the schedule management system 121 schedules the shift for the particular worker associated with the particular worker client device 106.

Referring next to FIG. 7B, shown is a flowchart that provides one example of the operation of a portion of the schedule management system 121 according to various embodiments. It is understood that the flowchart of FIG. 7B provides merely an example of the many different types of functional arrangements that may be employed to implement the operation of the portion of the schedule management system 121 as described herein. As an alternative, the flowchart of FIG. 7B may be viewed as depicting an example of elements of a method implemented in the computing environment 103 (FIG. 1) according to one or more embodiments.

In box 721, the schedule management system 121 receives an open shift opportunity from an employer client device 109 according to various embodiments of the present disclosure. The open shift opportunity may be received via an input on a user interface 180 rendered on the employer client device 109. In box 724, the schedule management system 121 identifies the requirements of the open shift. For example, the open shift opportunity may specify the skills, specialties, certifications, registrations, and/or other type of information that the employer is looking for in a worker to fill the open shift. In box 727, the schedule management system 121 via the matching engine 124 selects a first pool of user profiles based at least in part on the requirements of the open shift. For example, if the open shift is for a geriatric nurse, a user profile for a pediatric nurse may not be selected. Additionally, the employer can prefer to provide the open shift to its own employees within the network before providing the open shift to workers in the network that are not otherwise associated with the employer.

In box 730, the matching engine 124 ranks the user profiles according to a variety of factors such as for example, skills, experience, rating, location, worker similarities, feedback data, and/or any other factor as can be appreciated. In some embodiments, the factors are assigned weights and the sum of the weights may be used to rank the user profiles.

In box 733, notifications detailing the open shift can be generated and transmitted to the worker client devices 106 of the one or more top-ranked user profiles. In box 736, the schedule management system 121 determines whether a response has been received within a predefined threshold amount of time. If a response has been received from a particular worker client device 106 accepting the open shift, the schedule management system 121 proceeds to box 745. Otherwise, the schedule management system 121 proceeds to box 739. In box 739, the schedule management system 121 expands the pool of user profiles that can be considered for the open shift. As such, additional user profiles are considered, scored, and/or ranked. The schedule management system 121 transmits notifications of the open shifts to worker client devices 106 associated with the workers in the expanded pool of workers. In box 742, the schedule management system 121 receives an acceptance of the open shift from a particular worker client device 106. In box 745, the schedule management system 121 schedules the shift for the particular worker associated with the particular worker client device 106.

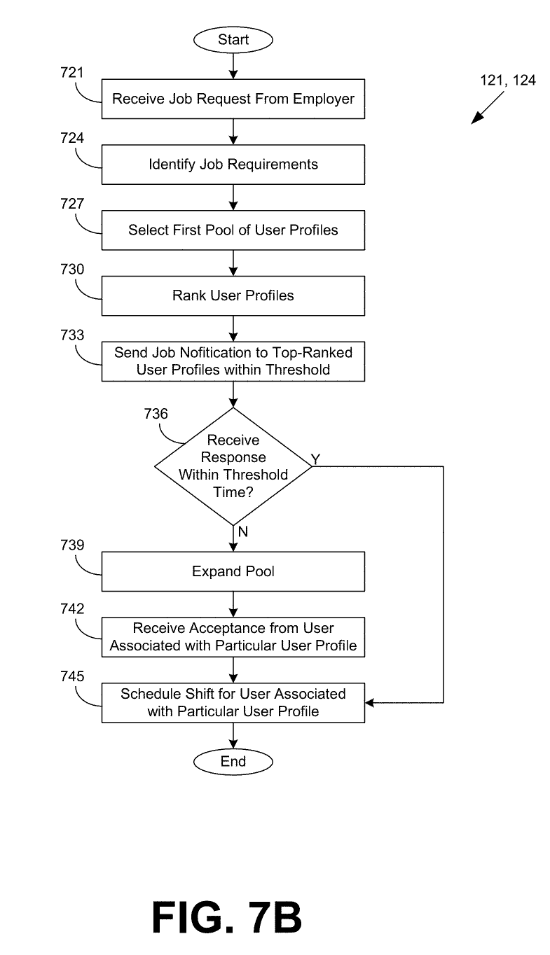

Referring next to FIG. 8, shown is a flowchart that provides one example of the operation of a portion of the schedule management system 121 according to various embodiments. It is understood that the flowchart of FIG. 8 provides merely an example of the many different types of functional arrangements that may be employed to implement the operation of the portion of the schedule management system 121 as described herein. As an alternative, the flowchart of FIG. 8 may be viewed as depicting an example of elements of a method implemented in the computing environment 103 (FIG. 1) according to one or more embodiments.

In box 800, the schedule management system 121 determines a start time of a scheduled shift for a worker. In box 803, the schedule management system 121 predicts a route of travel from the worker location to the shift location. In some embodiments, the worker location may be based on sending a request to the worker client device 106 for location data. In other embodiments, the worker location may be based on a residential address associated with the user profile and/or any other address provided by the worker client device 106. The shift location is determined according to the location of the shift as provided by the employer client device 109.

In some embodiments, the schedule management system 121 employs a third party navigation system (e.g., GOOGLE MAPS®) to determine a route between the two locations. In other embodiments, the schedule management system 121 can predict the route of travel according to prior user history. In box 806, the schedule management system 121 estimates a time for travel. For example, the schedule management system 121 may interface with a third party navigation system to determine the traffic information with the particular route. For example, if there is an accident or heavy traffic, the estimated time for travel may be longer than if there is not an accident or heavy traffic.

In box 809, the schedule management system 121 can send an alert notification to the worker client device notifying the worker of the estimated time for travel. The notification may be in the form of a text message, an email message, a push notification, and/or any other type of notification as can be appreciated that can be rendered on the worker client device. In some embodiments, the notification may include a preferred time of departure so that the worker can arrive at the shift location on time.

In box 812, the schedule management system 121 detects a departure of the worker client device 106 from the worker location. In some embodiments, the schedule management system 121 polls the worker client device 106 periodically for location data to determine when the worker client device 106 has left the worker location. In other embodiments, the worker client application 133 may detect a change in location and send a notification to the schedule management system 121 notifying the schedule management system 121 of the change in location. In box 815, the schedule management system 121 determines whether there is a change in the estimated time of arrival. For example, the schedule management system 121 may periodically interface with the third-party navigation system to determine whether there is a change in traffic between the worker location and the shift location. If there is a change in the time of arrival, the schedule management system 121 proceeds to box 821, and the schedule management system 121 generates and transmits a notification indicating the change in time to the employer client device 109. In box 818, the schedule management system 121 determines whether the worker client device 106 has arrived at the shift location. If the worker client device 106 has not arrived at the shift location, the schedule management system 121 proceeds to box 815. Otherwise, this portion of the schedule management system 121 ends.

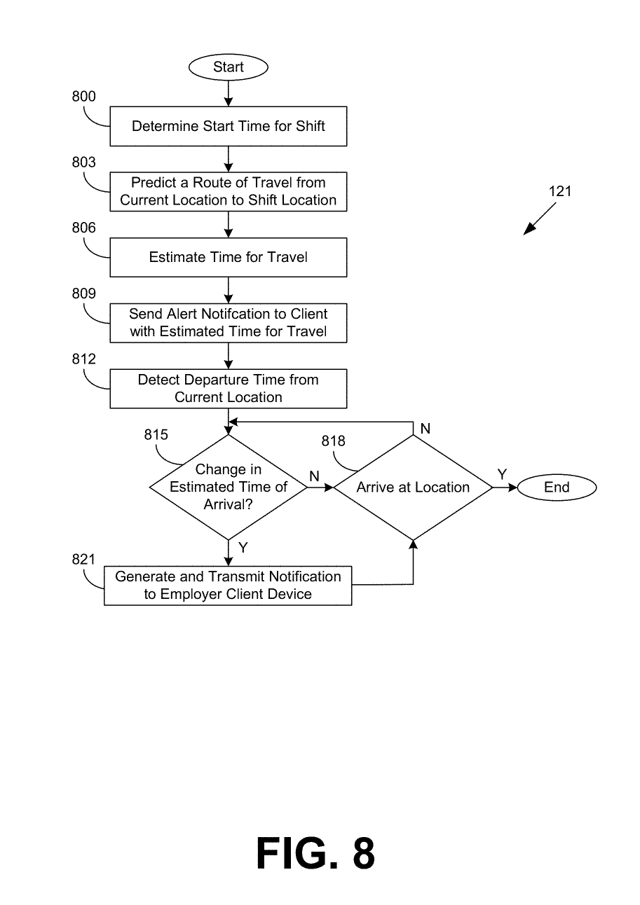

Referring next to FIG. 9, shown is a flowchart that provides one example of the operation of a portion of the schedule management system 121 according to various embodiments. It is understood that the flowchart of FIG. 9 provides merely an example of the many different types of functional arrangements that may be employed to implement the operation of the portion of the schedule management system 121 as described herein. As an alternative, the flowchart of FIG. 9 may be viewed as depicting an example of elements of a method implemented in the computing environment 103 (FIG. 1) according to one or more embodiments.

In box 900, the schedule management system 121 determines that a particular shift has ended. In some embodiments, the schedule management system 121 may determine that a shift has ended according to a scheduled end time associated with the shift. In some embodiments, the scheduled management system 121 may determine that a shift has ended according to a notification received via the worker client device 106 and/or the employer client device 109. In box 903, the schedule management system 121 sends a request for feedback to the employer client device 109. For example, the schedule management system 121 may generate a user interface 180 for rendering on the employer client device 109. The user interface 180 can comprise questions regarding the worker's performance during the shift. The user interface 180 can include components which can be used by the worker to input answers to the questions (e.g., drop down box, text entry box, button component, etc.). In some embodiments, the questions to be presented that are included in the user interface 180 can be selected from a pool of questions according to various factors such as, for example, type of worker, experience of worker, type of shift, etc.

At box 906, the schedule management system 121 receives the feedback from the employer client device 109. For example, the employer client device 109 can be configured to transmit the data inputted by an employer of the employer client device 109 via the user interface 180 to the schedule management system 121. At box 909, the schedule management system 121 updates the user profile according to the feedback. For example, the schedule management system 121 can update the skills, experience, ratings, and/or any other relevant data according to the feedback from the employer.

In one non-limiting example, the schedule management system 121 can assign weights to each of the answers of the questions. In some embodiments, a sum of the weights is determined and the schedule management system 121 can adjust the rating data for the worker according to the sum of the weights. For example, if the sum is below a threshold, the rating may be decreased by at least one value and if the sum meets or exceeds the threshold value, the rating may be increased by at least one value. In other embodiments, the rating data may be adjusted according to the weights of individual answers. After the user profile is updated, this portion of the schedule management system 121 ends.

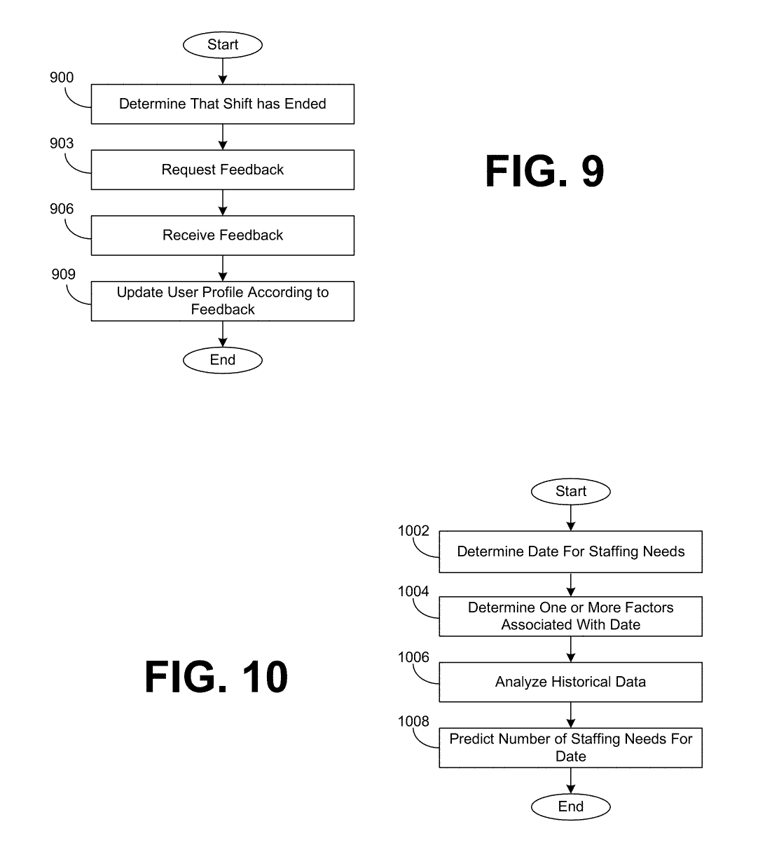

Referring next to FIG. 10, shown is a flowchart that provides one example of the operation of a portion of the schedule management system 121 according to various embodiments. It is understood that the flowchart of FIG. 10 provides merely an example of the many different types of functional arrangements that may be employed to implement the operation of the portion of the schedule management system 121 as described herein. As an alternative, the flowchart of FIG. 10 may be viewed as depicting an example of elements of a method implemented in the computing environment 103 (FIG. 1) according to one or more embodiments.

In box 1002, the schedule management system 121 determines a date for staffing needs according to various embodiments. For example, the schedule management system 121 may receive a particular date from the employer. In another example, the schedule management system 121 may automatically review future dates for particular hospitals. In some embodiments, the schedule management system 121 may determine a date based in part on an alert associated with a particular event (e.g., weather alert, emergency alert, etc.).

In box 1004, the schedule management system 121 determines one or more factors associated with the date. For example, the factors may include prior staffing needs, prior scheduling needs, weather conditions, seasons, shift cancellations, number of open shifts, day of the week, and/or any other factor as can be appreciated. In box 1006, the schedule management system 121 analyzes historical data associated with the employer and factors similar to those associated with the particular date. In box 1008, the schedule management system 121 employs machine learning models associated with the analysis of the historical data to predict a number of staffing needs for the particular date. Upon predicting a number of staffing needs, this portion of the schedule management system 121 ends.

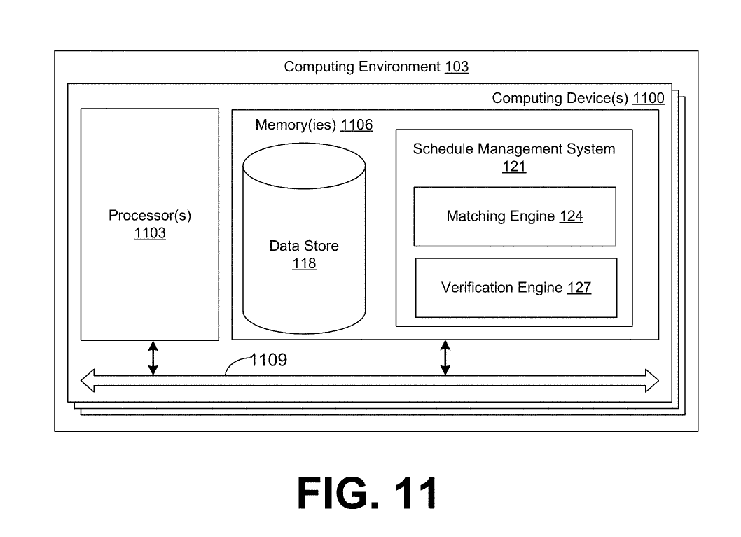

With reference to FIG. 11, shown is a schematic block diagram of the computing environment 103 according to an embodiment of the present disclosure. The computing environment 103 includes one or more computing devices 1100. Each computing device 1100 includes at least one processor circuit, for example, having a processor 1103 and a memory 1106, both of which are coupled to a local interface 1109. To this end, each computing device 1100 may comprise, for example, at least one server computer or like device. The local interface 1109 may comprise, for example, a data bus with an accompanying address/control bus or other bus structure as can be appreciated.

Stored in the memory 1106 are both data and several components that are executable by the processor 1103. In particular, stored in the memory 1106 and executable by the processor 1103 are the schedule management system 121, the matching engine 124, the verification engine 127, and potentially other applications. Also stored in the memory 1106 may be a data store 118 and other data. In addition, an operating system may be stored in the memory 1106 and executable by the processor 1103.

It is understood that there may be other applications that are stored in the memory 1106 and are executable by the processor 1103 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1106 and are executable by the processor 1103. In this respect, the term “executable” means a program file that is in a form that can ultimately be run by the processor 1103. Examples of executable programs may be, for example, a compiled program that can be translated into machine code in a format that can be loaded into a random access portion of the memory 1106 and run by the processor 1103, source code that may be expressed in proper format such as object code that is capable of being loaded into a random access portion of the memory 1106 and executed by the processor 1103, or source code that may be interpreted by another executable program to generate instructions in a random access portion of the memory 1106 to be executed by the processor 1103, etc. An executable program may be stored in any portion or component of the memory 1106 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1106 is defined herein as including both volatile and nonvolatile memory and data storage components. Volatile components are those that do not retain data values upon loss of power. Nonvolatile components are those that retain data upon a loss of power. Thus, the memory 1106 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1103 may represent multiple processors 1103 and/or multiple processor cores and the memory 1106 may represent multiple memories 1106 that operate in parallel processing circuits, respectively. In such a case, the local interface 1109 may be an appropriate network that facilitates communication between any two of the multiple processors 1103, between any processor 1103 and any of the memories 1106, or between any two of the memories 1106, etc. The local interface 1109 may comprise additional systems designed to coordinate this communication, including, for example, performing load balancing. The processor 1103 may be of electrical or of some other available construction.

Although the schedule management system 121, the matching engine 124, the verification engine 127, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts of FIGS. 4-10 show the functionality and operation of an implementation of portions of the schedule management system 121, the matching engine 124, and the verification engine 127. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 1103 in a computer system or other system. The machine code may be converted from the source code, etc. If embodied in hardware, each block may represent a circuit or a number of interconnected circuits to implement the specified logical function(s).

Although the flowcharts of FIGS. 4-10 a specific order of execution, it is understood that the order of execution may differ from that which is depicted. For example, the order of execution of two or more blocks may be scrambled relative to the order shown. Also, two or more blocks shown in succession in the flowcharts of FIGS. 4-10 may be executed concurrently or with partial concurrence. Further, in some embodiments, one or more of the blocks shown in FIGS. 4-10 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the schedule management system 121, the matching engine 124, and the verification engine 127, that comprises software or code can be embodied in any non-transitory computer-readable medium for use by or in connection with an instruction execution system such as, for example, a processor 1103 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the schedule management system 121, the matching engine 124, and the verification engine 127, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 1100, or in multiple computing devices 1100 in the same computing environment 103.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A system, comprising:
at least one computing device; and
a schedule management application executable in the at least one computing device, wherein when executed the schedule management application causes the at least one computing device to at least:
obtain user profile information of a user from a series of user interfaces sequentially rendered on a client device, the user profile information comprising contact information, skills information, experience information and credential information;
automatically establish a connection with a third-party device to automatically verify at least a portion of the user profile information; and
upon verification, automatically establish a user account for the user to allow the user to be considered for a plurality of open shifts available via a plurality of healthcare workplaces.

2. The system of claim 1, wherein the client device is a mobile device.

3. The system of claim 1, wherein, when executed, the schedule management application further causes the at least one computing device to at least:
determine that the user profile information includes an image;
extract profile data from the image; and
store the profile data in a data store with respect to the user account.

4. The system of claim 3, wherein extracting the profile data comprises at least:
converting the image to a readable format;
parsing the readable format; and
identifying at least one term associated with at least one predefined data field based at least in part on a plurality of predefined keywords, the at least one term corresponding to the profile data.

5. The system of claim 1, wherein obtaining user profile information further comprises:
establishing a connection with a social media system;
requesting the user profile information associated with a social media account of the user; and
receiving the user profile information associated with the social media account from the social media system.

6. The system of claim 1, wherein the user accumulates one or more incentives in response to the user entering user profile information in at least a portion of the series of user interfaces are sequentially rendered.

7. A system, comprising:
at least one computing device; and
a schedule management application executable in the at least one computing device, wherein when executed the schedule management application causes the at least one computing device to at least:
identify an open shift associated with a workplace, the open shift being based at least in part on open shift information received from an employer client device associated with the workplace;
identify one or more requirements associated with the open shift;
select a subset of a plurality of user profiles based at least in part on the one or more requirements;
rank individual user profiles in the subset of the plurality of user profiles according to a plurality of factors, the plurality of factors comprising at least one of: one or more skills, one or more specialties, user experience, a certification level, at least one user preference, at least one employer preference, a type of workplace associated with the open shift, a distance between a user location and a workplace location, or a respective calendar associated with the individual user profiles;
select at least one user profile from the subset of the plurality of user profiles based at least in part on a ranking of the at least one user profile;
generate a notification including a request to schedule the open shift; and
transmit the notification to at least one client device associated with the at least one user profile.

8. The system of claim 7, wherein the at least one user profile is within a first pool of user profiles selected, and when executed, the schedule management application further causes the at least one computing device to at least:
determine that a response to the notification has not be received from the at least one client device within a predefined threshold amount of time;
expand the first pool of user profiles to include a second pool of user profiles; and
transmit the notification to respective client devices associated with the second pool of user profiles.

9. The system of claim 7, wherein, when executed, the schedule management application further causes the at least one computing device to at least:
receive a schedule request for the open shift from a particular client device via a user interface rendered by the particular client device; and
schedule the open shift with a user associated with the particular client device.

10. The system of claim 9, wherein, when executed, the schedule management application further causes the at least one computing device to at least:
generate data encoding a feedback user interface including a plurality of feedback questions associated with a performance of the user;
transmit the data encoding the feedback user interface to the employer client device following completion of the open shift by the user; and
update the user profile associated with the user in response to receiving a plurality of answers to the plurality of the feedback questions.

11. The system of claim 9, wherein, when executed, the schedule management application further causes that at least one computing device to at least:
determine a route between the user location and the workplace location;
determine an estimated time of travel between the user location and the workplace location based at least in part on at least one of the route and current traffic associated with the route; and
send an alert to the particular client device including the estimated time of travel.

12. The system of claim 11, wherein, when executed, the schedule management application further causes the at least one computing device to at least:
determine a new user location based at least in part on location data received from the particular client device; and
update the estimated time of travel based at least in part on the new user location.

13. The system of claim 12, wherein, when executed, the schedule management application further causes the at least one computing device to at least:
determine that the user will arrive at the workplace location at a time after a start time of the open shift based at least in part on the estimated time of travel and updated traffic associated with the route; and
send a notification to the employer client device notifying an employer that the user will arrive late.

14. The system of claim 7, wherein, when executed, the schedule management application further causes the at least one computing device to at least:
obtain user profile information for individual user profiles from a series of user interfaces sequentially rendered on a respective client device associated with the individual user profiles, the user profile information comprising contact information, skills information, experience information and credential information.

15. A method, comprising:
establishing, via at least one computing device, a plurality of user accounts associated with a plurality of users, the plurality of user accounts being established for consideration for a plurality of open shifts associated with a plurality of workplaces;
matching, via at least one computing device, at least one user account with at least a subset of the plurality of open shifts;
generating, via at least one computing device, a notification including a request to schedule at least one open shift of the at least the subset of the plurality of open shifts;
transmitting, via the at least one computing device, the notification to at least one client device associated with the at least one user account; and
scheduling, via the at least one computing device, a particular open shift with a particular user associated with a particular client device in response to receiving a selection of the request from the particular client device based at least in part on the notification.

16. The method of claim 15, wherein establishing the plurality of user accounts comprises:
for individual user accounts of the plurality of user accounts,
obtaining, via the at least one computing device, user profile information of a respective user from a series of user interfaces sequentially rendered on a respective client device, the user profile information comprising at least one of contact information, skills information, experience information, and credential information;
establishing, via the at least one computing device, a connection with a third-party device over a network; and
verifying, via the at least one computing device, an accuracy of the user profile information in response to data received via the third-party device.

17. The method of claim 16, wherein obtaining user profile information further comprises:
receiving, via the at least one computing device, a document from the respective client device;
determining, via the at least one computing device, that the document includes an image; and
extracting the user profile information from the image.

18. The method of claim 17, wherein extracting the user profile information comprises at least:
converting, via the at least one computing device, the image to a readable format;
parsing, via the at least one computing device, the readable format; and
identifying, via the at least one computing device, at least one term associated with at least one predefined data field based at least in part on a plurality of predefined keywords, the at least one term corresponding to the user profile information.

19. The method of claim 18, further comprising:
generating, via the at least one computing device, a respective score for the individual user accounts, the respective score being based at least in part on at least one of: one or more skills, one or more specialties, user experience, a certification level, at least one user preference, at least one employer preference, a type of workplace associated with the open shift, a distance between a user location and a workplace location, or a respective calendar associated with the individual user accounts; and
ranking, via the at least one computing device, the plurality of user accounts according to the respective score.

20. The method of claim 16, further comprising identifying at least one of an extra skill, an extra specialty, or an extra user experience not provided by the respective user based at least in part on at least one of a previously received feedback of the respective user.
